(19) United States
(12) Patent Application Publication
(10) Pub. No.: US 2016/0136205 A1
(43) Pub. Date: May 19, 2016

(54) TARGETING TYPE I NKT CELLS TO CONTROL INFLAMMATION

(71) Applicant: Charles T. Spencer, El Paso, TX (US)

(72) Inventor: Charles T. Spencer, El Paso, TX (US)

(73) Assignee: The Board of Regents of the University of Texas System, Austin, TX (US)

(21) Appl. No.: 14/941,727

(22) Filed: Nov. 16, 2015

Related U.S. Application Data
(60) Provisional application No. 62/080,950, filed on Nov. 17, 2014.

(51) Int. Cl. A61K 35/17; A61K 38/20

(52) U.S. Cl. CPC A61K 35/17; A61K 38/20; A61K 38/2033; A61K 38/2066; A61K 38/2086; A61K 38/2026

(57) ABSTRACT

Embodiments are directed to methods and compositions for modulating cytokine responses in subject infected by a pathogen.

This application claims priority to U.S. Provisional Application Ser. No. 62/080,950 filed Nov. 17, 2014, which is incorporated herein by reference in its entirety.

### BACKGROUND

Francisella tularensis, the causative agent of tularemia, triggers an excessive response from the host immune response causing multiple organ failure and death. This bacterium is endemic to many animal species and can survive in many environmental conditions, including soil and water. It remains infectious even after an infected animal has decayed, thereby prolonging the risk of exposure. It can be transmitted to humans by direct contact with infected dead animal carcasses, inhalation of the bacteria from infected animals or the soil, ingestion of contaminated meats, or bites from infected insects, primarily ticks and mosquitoes. Because F. tularensis is highly infectious, it is a public health threat and a potential terrorist bioweapon.

Exposure to F. tularensis results in excessive inflammation and the release of a storm of proinflammatory host molecules. This host “cytokine storm” is responsible for the severity of symptoms and ultimately for the death of the host. Regulatory cells of the immune system normally work to prevent inflammation and this life-threatening cytokine storm. However, death from this disease results when the inflammation and the cytokine storm overwhelm the regulatory cells. Of great concern in the treatment of tularemia is that antibiotics do not address this cytokine storm, which is the root cause of death from this organism. In addition, other infectious diseases initiate cytokine storms and suffer from a lack of treatment options. There remains a need for a method to treat tularemia and other infectious diseases that can induce cytokine storms in an infected animal.

### SUMMARY

The inventor contemplates that controlling, attenuating, or modulating the overly robust cytokine response to a pathogen (“cytokine storm”) can prevent or lower the risk of death. Natural killer T cells (NKT), a population of white blood cells displaying cytokine regulatory functions, can be manipulated and/or modulated for controlling the cytokine storm. NKT cells regulate the cytokine storm in animals that survive Francisella tularensis infection and regulatory function can be elicited or enhanced in hosts to prevent disease. Cell culture systems are used investigate the mechanism by which NKT cells control the cytokine storm. Other studies include animal models to compare the cytokine storm response in infected mice that have an adequate NKT response and those that lack these mechanisms. The compositions and methods described here can be used not only for F. tularensis treatment, but can be broadly applied to other infectious diseases that elicit the life-threatening cytokine storm (e.g., pandemic influenza). In certain aspects NKT cells are isolated from an individual, expanded in vitro, conditioned in vitro, and the conditioned NKT cells administered to the subject. In certain aspects the NKT cell is an autologous cell. In other aspects the NKT cell can be a transplant from a compatible donor. In still a further aspect the NKT cell can be an engineered/recombinant cell. In other embodiments the subject can be administered an NKT modulating agent. In certain aspects the modulating agent is IL-4, IL-5, IL-10, IL-13, OR IL-21 or any combination therein. In certain aspects the NKT modulating agent is αGalCer, αGalDAG or variants thereof.

NKT cells are able to respond rapidly to infection and inflammation by producing regulatory or inflammatory cytokines to control innate and adaptive immune responses or directly lysing infected cells (FIG. 1). In addition, NKT cells provide both antimicrobial and regulatory effector functions that could control the immune response while simultaneously attacking the infecting pathogen. The possibility of targeting NKT cells for therapeutic benefit is being pursued in other diseases that elicit inflammation, e.g., cancer and influenza, with conflicting results. In addition, NKT cells produce IFN-γ 6 to 24 h after infection by F. tularensis indicating a direct involvement in the response. However, it is unknown whether this IFN-y production by NKT cells contributes to the cytokine storm or whether the regulatory effector functions of type I or type II NKT cells are elicited to suppress F. tularensis-induced inflammation.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. NKT cells are rapidly activated following infection (within 6 to 24 hours) and are capable of producing regulatory, pro-inflammatory and cytotoxic effector functions depending upon the antigenic stimuli.

FIG. 2. Infection with F. tularensis activates NKT cells. Two mice were inoculated with LVS and their spleens resected 12 and 24 hours later. NKT cells were identified as B220⁻, NK1.1+, CD3_{ε}+ and activation determined by CD69 stain.

FIG. 3. NKT cell-deficient mice are more susceptible to F. tularensis infection. C57BL/6J and CD1d-/- mice were inoculated with 1×10⁶ cfu Ft LVS. Weight loss and symptoms of disease were monitored daily for 12 days. Significantly more NKT cell-deficient mice succumbed to the infection than their wild type counterparts (*p=0.0016).

FIG. 4. NKT cell-deficient mice generate a stronger cytokine storm. Animals were inoculated with 1×10⁶ cfu Ft LVS. Blood samples were obtained individually 3 dpi and at the time of each mouse's sacrifice (4-6 dpi) for comparison with pre-immune samples. *p<0.05, n=5-12.

FIG. 5. iNKT cell-deficient mice are more susceptible to F. tularensis infection. B6 and B6-Jα18-/- mice were inoculated with 1×10⁶ cfu Ft LVS. Weight loss and symptoms of disease were monitored daily for 12 days. Significantly more NKT cell-deficient mice succumbed to the infection than their wild type counterparts (p=0.004).

FIG. 6. NKT cells suppress cytokine production in vitro. Macrophages were infected with LVS for 2 hours before purified NKT cells were added. IL-6 and IL-1β secretion into the supernatant was measured by ELISA after 48 hours. (p=0.0106)

FIG. 7. Bacterial burden in the liver and spleen is not dependent on NKT cells. Liver and spleen were isolated from B6 or B6-CD1d-/- LVS-inoculated mice. Bacterial burden was determined by plating tissue homogenate on modified Mueller-Hinton plates.

FIG. 8. NKT cells inhibit intracellular growth of F. tularensis. Inhibition of intracellular F. tularensis growth was observed when infected macrophages were culture with purified NKT cells (p=0.1). Bacteria released from lysed macrophages were grown in broth containing H3-uridine O/N and was measured using scintillation counting (solid bars, left axis). Bacteria outgrowth was determined on SBA plates (hashed bars, right axis).

FIG. 9A-B. NKT cell suppression of IL-6 (A) is reversed by addition of neutralizing antibodies against IL-21, IL-5, IL-13, and potentially IL-4 and IL-10 (B). Addition of neutralizing antibodies alone or the isotype control did not result in reversal of inhibition.

### DESCRIPTION

Despite their damaging side-effects, cytokines and chemokines play a vital role as signaling molecules to recruit and activate immune lymphocytes. Inflammation produces both pro-inflammatory cytokines to activate the immune response and anti-inflammatory cytokines to dampen it. The production of anti-inflammatory cytokines is crucial for the self-limiting resolution of the inflammatory state and prevention of the cytokine storm. It is unclear why, for pathogens such as F. tularensis, inflammation escapes immune control and causes host pathology. It is possible that a specific combination of cytokines produced for a specified time is required for this destructive inflammation-mediated cytokine storm.

Inflammation is being increasingly recognized as contributing, in varying degrees, to the pathology of several infectious diseases, of which F. tularensis is an example. F. tularensis, the causative agent of tularemia, is a highly infectious bacterium, and potential bioweapon, endemic to the US. Francisella tularensis infection triggers an exuberant host inflammatory response defined by excess production of pro-inflammatory cytokines by immune cells, a condition termed “cytokine storm.” Current understanding is that inflammation and the subsequent cytokine storm causes the totality of the pathology associated with tularemia including the death of the host. Since antibiotics do not prevent this cytokine storm and its associated tissue damage, new therapeutics are needed to treat inflammatory infectious diseases.

Natural killer T (NKT) cells participate in host defense against infections by recognizing microbial lipids. There are two subtypes of NKT cells, type I and type II, based on receptor expression and antigen recognition. Type II NKT cells have traditionally been implicated in suppressing inflammation. Type I, or invariant, iNKT cells are generally considered cytotoxic although, there is emerging data that they too may suppress inflammation under certain conditions. Despite a single report of NKT cell activation during F. tularensis infection, the contribution of either of these subsets of NKT cells to protection from F. tularensis-mediated death remains undefined

Inflammation is initiated upon infection or tissue damage as the host's first line of defense. The molecular processes of inflammation activate both the innate and adaptive immune responses to contain and eliminate the threat. Most microbes, therefore, have evolved various strategies to avoid triggering the inflammatory response, contrary to the robust activation of inflammation elicited by F. tularensis.

Inflammation was previously considered to be limited to minor, localized insults, whereas bacterial infections of the blood led to septicemia. However, it is now recognized that over-production of pro-inflammatory cytokines from infections localized to tissues can also lead to systemic inflammation. Indeed, localized infections by influenza and F. tularensis trigger life-threatening inflammation that results in massive release of pro-inflammatory cytokines and chemokines into the blood, termed a “cytokine storm.”

Waves of cytokines in the bloodstream have been identified for many infectious diseases, which indicate the induction of inflammation. However, for most of these diseases, the waves of cytokines is limited in intensity, duration or both and the composition varies between diseases. For diseases like tularemia, the prolonged intense cytokine storm results in severe pathology. Lethal infection causes inflammation-mediated deterioration of liver and spleen. The F. tularensis-induced cytokine storm causes edema, depressed blood volume and pressure, septic shock, and multiple organ system failure.

The virulent Type A Francisella tularensis subspp. tularensis (a NIH Category A Priority Pathogen) is a gram-negative facultative intracellular coccobacillus and the causative agent of the lethal zoonotic disease tularemia in humans. Though infection with F. tularensis is primarily airborne, it can also be acquired through ingestion of contaminated meats or by insect bites, primarily tick and mosquito. Because of its airborne transmission, environmental stability and high infectivity, F. tularensis is considered a potential bioweapon and public health concern.

An infectious dose of 10 to 50 cfu F. tularensis subspp. tularensis acquired subcutaneously or by inhalation is sufficient to cause lethal disease in humans. In mice, a single bacterium is sufficient to cause lethal disease following intraperitoneal inoculation. Due to the virulence of F. tularensis subspp. tularensis, much of our understanding of tularemia comes from studies of the attenuated F. tularensis subspp. holartica-derived Live Vaccine Strain (F. tularensis LVS). Although F. tularensis LVS causes little or no disease in humans, it recapitulates the tularemia disease well in mice.

Unlike many bacterial infections, inflammation is not elicited until the bacterium has infected its target cell as the F. tularensis lipopolysaccharide (LPS) is not recognized by TLR4. F. tularensis infection begins upon uptake of the bacterium by macrophages into the phagolysosome. Escape of the bacterium into the cytosol initiates inflammation via caspase 1-dependent inflammasome-mediated secretion of IL-1β and IL-18. During lethal infection, excessive levels of IFN-γ and IL-6 are observed in the serum peaking three to five days post inoculation. Nonetheless, it is well established that immunity to F. tularensis critically depends upon eliciting those same inflammatory cytokines, e.g., IL-12, IFN-γ and TNF-α. Therefore, to protect against the cytokine storm, inflammation must be dampened without completely eliminating the innate immune response.

Therapeutic treatment of the pathogen-induced cytokine storm could be accomplished by either eliminating the infecting pathogen or directly reducing inflammation. Eliminating the pathogen with antibiotics or antivirals removes the inducer of inflammation but requires time before natural waning of the cytokine storm. In addition, due to the lack of promising antibiotic and antiviral therapies in development and the potential for drug-resistant strains, alternative and more rapidly acting approaches targeting the host immune response are being considered. For infectious diseases such as tularemia, where a dysregulated immune response causes significant damage, suppressing the cytokine storm may allow for a more direct and robust alternative therapy.

To date, however, efforts using a variety of anti-inflammatory drugs and adjunct therapies to target the cytokine storm have proven unsuccessful. One of the challenges of therapeutically targeting the inflammatory response is its critical participation in clearing infection. Unlike therapies designed to kill off the pathogen, the inflammatory response cannot be completely eliminated. Complete elimination of inflammation would result in no activation of the immune response and failed clearance of the infecting organism. However, therapies that target immune regulatory cells may provide suppression without completely eliminating the inflammatory response.

Immune regulatory cells, including NKT cells, T_{reg} cells and myeloid-derived suppressor cells (MDSCs), are naturally activated during the inflammatory response in order to prevent self-inflicted damage. NKT cells have been reported to regulate the inflammatory response through activation of both T_{reg} and MDSCs. This indicates NKT cells control other immune cells and represent a central point for development of novel therapeutics.

In addition, recent studies and observations support this assertion. First, cells expressing shared NK and NKT cell markers were shown to be increased in mice with severe F. tularensis infection. Secondly, co-activation of NK and NKT cells by IL-15 administration reduced F. tularensis-induced pro-inflammatory cytokine production in tissues. In addition, IL-15-deficient mice have impaired survival in response to F. tularensis infection.

NKT cells are divided into two subsets depending upon their TCR expression and lipid reactivity. Type I iNKT cells express a semi-invariant TCR comprised of the mouse Vα14/Jα18 rearrangement paired with Vβ8.2, Vβ7, or Vβ2.37, Type I iNKT cells recognize glycolipid antigens presented in context of the MHC-like molecule, CD1d. Host (iGb3),43 microbial (GSL1-4, αGDAG) and synthetic (αGalCer, OCH) lipids have been isolated which stimulate type I iNKT cells. The αGalCer lipid is a highly potent agonist originally isolated from a marine sponge that is recognized by all type I iNKT cells.

Type II NKT cells display a more heterogeneous TCR usage. These cells recognize different CD1d-restricted lipids than type I iNKT cells, such as sulfatide and lysophosphatidylcholine, and do not recognize αGalCer. Studies suggest that type II NKT cells generally have a regulatory role under conditions of inflammation and are antagonistic to pro-inflammatory iNKT cells. However, emerging data suggest that, under certain conditions, type I iNKT cells can also suppress inflammation.

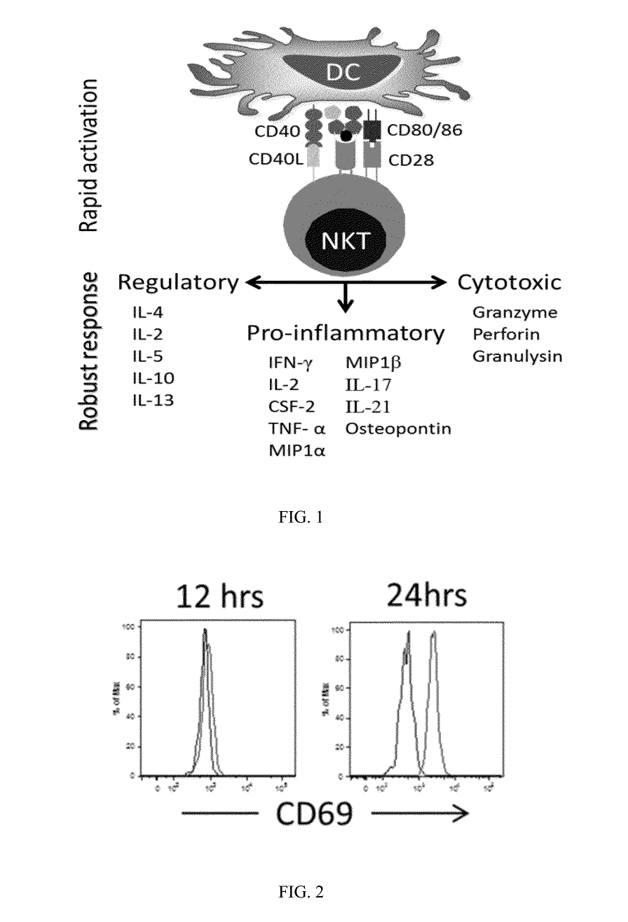

NKT cells are activated within 24 hours after F. tularensis inoculation and, depending upon the antigenic stimulus received, can produce robust cytotoxic or regulatory or pro-inflammatory cytokine responses (FIG. 1). Due to their rapid activation and robust tunable response, NKT cells may play a central role in controlling F. tularensis-induced inflammation and cytokine storm.

### I. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

1. Determine whether one or both NKT cell subsets enhance survival after F. tularensis exposure. Type I and type II NKT cells both express NK1.1, TCRβ, CD3 and can be either CD4⁺ or CD4, CD8 double negative. The single report demonstrating NKT cell activation following F. tularensis infection classified NKT cells as NK1.1⁺, TCRβ⁺, CD3⁺ which will not distinguish between type I and type II NKT cells.¹⁵ However, these subsets can be distinguished ex vivo based upon their antigen recognition. All type I iNKT cells recognize αGalCer-loaded CD1d-tetramers (αGalCer-tetramer) whereas sulfatide-loaded CD1d-tetramers (sulfatide-tetramer) label a broad range of type II NKT cell specificities.⁵⁹

1a. Determine which NKT cell subset(s) are activated in response to F. tularensis infection. B6 mice will be inoculated with F. tularensis LVS and groups of 3 mice sacrificed at 6, 12, 24, and 36 hours post inoculation along with 2 uninfected controls. The activation of spleen and liver resident NKT cells will be measured by multiparametric flow cytometry. Type I iNKT cells are defined as B220⁻, CD3ε⁺, NK1.1⁺, αGalCer-tetramer⁺; and type II NKT cells are defined as B220⁻, CD3ε⁺, NK1.1⁺, sulfatide-tetramer⁺. In addition, B220⁻, CD3ε⁺, NK1.1⁺, αGalCer-tetramer⁻, sulfatide-tetramer⁻ cells will be considered as type II NKT cells not recognizing sulfatide. Activation will be observed by upregulation of CD69 surface expression (FIG. 2) and production of IFN-γ or IL-4.⁶⁰

Activated tetramer⁺ NKT cells will be further characterized by their expression of CD4, CD24, NK1.1 and IL-25R. Stages of NKT cell maturation are defined by the temporal expression of CD24 and NK1.1 with expression of NK1.1 indicating terminal maturation for both CD4⁺ and CD4⁻ subsets.⁶⁰ Differential cytokine production has been documented by NKT cell populations, particularly the ability of CD4⁻, NK1.1⁻ cells to produce IL-17.⁶¹ In addition, IL-25R⁺, CD4⁺ NKT cells have been shown to produce large amounts of IL-4 and IL-13 but little IFN-γ.⁶² In addition, cytotoxic NKT cells express perforin, granzyme A, granzyme B, and granulysin. It is important, therefore, to understand which NKT cell subsets are activated and what effector functions they produce following F. tularensis infection. These studies define the phenotype of NKT cells activated in response to F. tularensis infection.

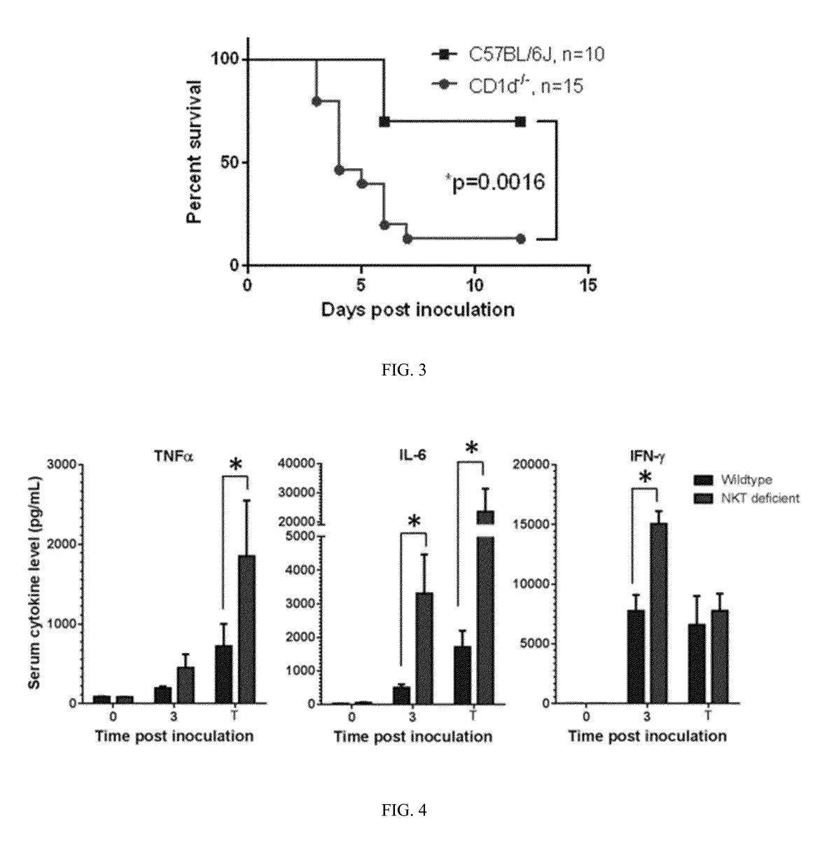

1b. Determine whether one or both NKT cell subsets prevent the cytokine storm and enhance survival. To date, no reports have directly investigated the role of NKT cells in enhancing survival after F. tularensis exposure. Mice deficient in CD1d, the developmental positive selecting antigen presenting ligand, do not develop NKT cells. Therefore, the survival of wild type B6 and B6-CD1d^{-/-} mice can be compared after F. tularensis LVS challenge. Mice deficient in NKT cells had poorer survival compared with NKT cell sufficient mice (FIG. 3). Furthermore, blood monitoring of serum cytokine levels revealed that NKT cell-deficient mice developed a significantly stronger inflammatory response as indicated by the increased levels of TNF-α, IL-6 and IFN-γ (FIG. 4).

However, CD1d-deficient mice lack both subsets of NKT cells. Therefore, in order to analyze the function of type I iNKT cells in vivo, the B6-Jα18^{-/-} mouse strain can be used. Mice deficient in the TCR-Jα18 gene segment cannot generate the invariant type I iNKT cell receptor while type II NKT cell development remains intact.¹⁰ Therefore, by comparing survival and the development of the cytokine storm in Jα18^{-/-} mice with wild type and CD1d^{-/-} mice, the contribution of type I iNKT cells in enhancing survival to F. tularensis challenge in vivo can be assessed.

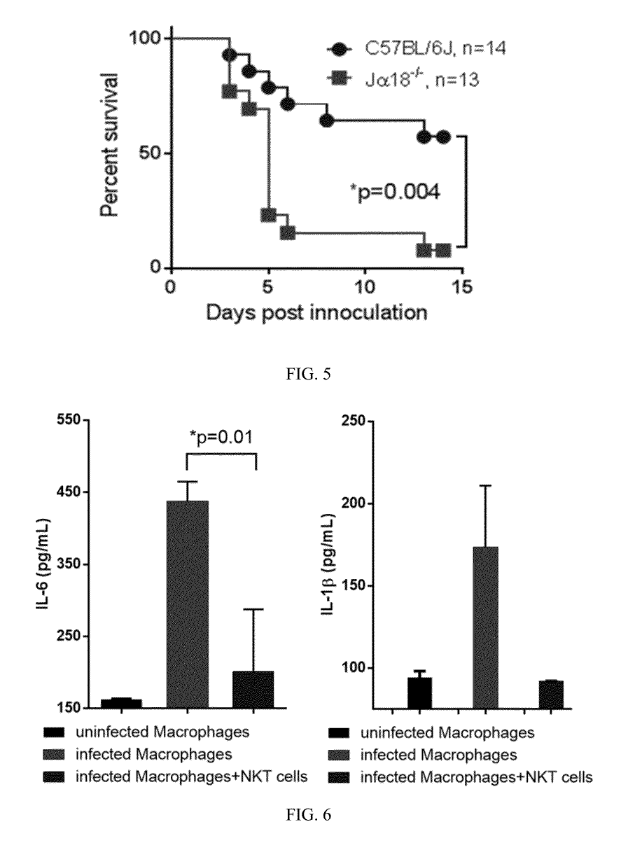

Data implicates type I iNKT cells in enhancing survival of mice inoculated with F. tularensis LVS (FIG. 5). Jα18^{-/-} mice displayed reduced survival compared with wild type controls (p=0.004). Their survival is comparable to that of CD1d^{-/-} mice suggesting that perhaps type I iNKT cells may be involved in controlling F. tularensis-induced inflammation.

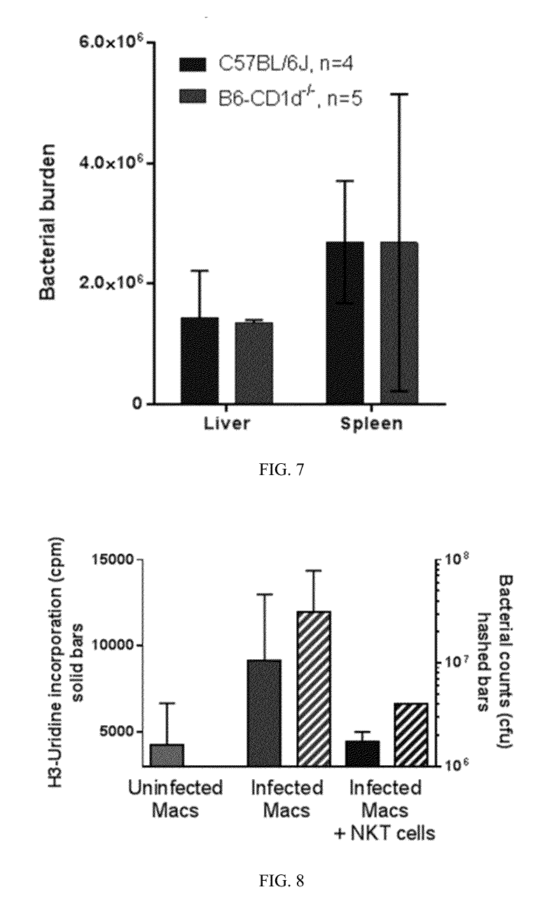

Due to their heterogeneity, no genetic animal model currently exists that is specifically deficient in type II NKT cells. However, most type II NKT cells recognize the sulfatide-tetramer.⁵⁹ Therefore, the toxin saporin conjugated to the sulfatide-tetramer will be utilized to deplete type II NKT cells in vivo prior to challenge.^{63,64} Saporin is a ribosome-inactivating protein that is normally cell impermeable. However, when conjugated to the sulfatide-tetramers, it will be specifically targeted to type II NKT cells where it is internalized. Subsequent degradation of the tetramer releases saporin, which halts protein translation thereby killing the cell. Saporin conjugated to αGalCer-tetramers have been used to deplete type I iNKT cells, thereby demonstrating the feasibility of this procedure. Splenic type I iNKT cells were reduced to 85% by 3 days after injection (FIG. 6) but began to repopulate within 5 days.

To deplete type II NKT cells, 66.5 pM sulfatide-tetramer-toxin conjugate will be injected into B6 mice two days prior to F. tularensis LVS inoculation. This time point was chosen since NKT cells are activated one day after inoculation (see FIG. 2). Therefore, administration of the tetramer-toxin will occur three days prior to NKT cell activation allowing for maximal depletion before F. tularensis exposure. Since repopulation of the spleen five days after treatment has been observed, a second dose of the tetramer-toxin will be given four days after the first dose to maintain the depletion of the population. If needed, a third dose can be given four days later; however, those mice that survive to this time are generally recovering from the infection already. Control groups will receive injections of the non-conjugated toxin or non-conjugated tetramer at the same time points.

Infection procedure: 25 to 30 g mice will be inoculated intradermally with 1×10⁶ cfu F. tularensis LVS (LD₅₀ for C57BL/6J) and their morbidity and mortality followed daily over the subsequent 12 days. Animals will be weighed daily and considered terminal after having lost 20% of their initial body weight. In addition, animals will be monitored daily for clinical signs (CS) of illness and scored according to the severity of their symptoms, where: 0=healthy, 1=slight ruffled fur, 2=increase ruffled fur or hunched posture, 3=both severely ruffled fur and hunched posture, and 4=lethargic/immobile. Animals will be monitored up to four times daily in order to avoid natural death of the animals and considered terminal when they reach CS=3-4. Animals with CS=2 having lost 20% of their body weight also will be considered terminal and euthanized. However, animals that lose 20% of their body weight also graded CS=3-4.

Blood samples (˜30 μL) will be taken on days 1, 3, 5, and 7 to measure the intensity of the cytokine storm. At the time of sacrifice, blood will be collected by heart puncture and the spleen, liver and lungs will be dissected. A segment of each organ will be homogenized and serial dilutions plated on modified Mueller-Hinton plates supplemented with 5% sheep blood and 1% isovitalex to determine the bacterial load. Supernatants from this tissue homogenate will be used for gross cytokine analysis. The rest of the organ will be archived for future histological sectioning and/or RNA harvesting for real-time RT-PCR.

Serum will be isolated from blood samples and the cytokine content measured by multiplex ELISA. 14 to 20 cytokines are analyzed in individual 10 μL blood samples. This allows the analysis of each time point for each mouse independently. This analysis will reveal the development of the cytokine storm following F. tularensis inoculation. Comparisons between wild type, CD1d^{-/-}, Jα18^{-/-} and sulfatide-tetramer-toxin-treated mice will allow us to determine the role of NKT cell subsets in suppressing inflammation and the cytokine storm. These experiments will be repeated three times with 15 mice per group to yield statistically meaningful results.

Decreased survival and loss of suppression of the cytokine storm in NKT cell-deficient mice would indicate the involvement of these NKT cells. Similar survival between CD1d^{-/-} and Jα18^{-/-} mice will be interpreted as a predominant role for type I iNKT cells in enhancing survival. Conversely, similar survival between CD1d^{-/-} and sulfatide-tetramer-toxin-treated mice would suggest a predominant role for type II NKT in enhancing survival. Intermediate survival of both groups of mice singly deficient in NKT cell subsets between wild type and CD1d^{-/-} mice will be interpreted as evidence for participation by both NKT cell subsets in enhancing survival after F. tularensis challenge.

1c. Reconstitute NKT cell-deficient mice with individual NKT cell subsets. Type I and Type II NKT cells will be individually purified by FACS from the spleens of B6 mice based upon their recognition of αGalCer-tetramer or sulfatide-tetramer, respectively.⁶⁰ Purified NKT cells will be adoptively transferred into Jα18^{-/-} mice to reconstitute the system.³³ For this, 1−2×10⁶ NKT cells will be injected into the tail vein of 15 Jα18^{-/-} mice. Following a 2 hour rest, mice will be challenged with F. tularensis LVS and their survival and inflammation monitored over the next 12 days. Upon sacrifice, the degree of NKT cell-reconstitution in the spleen and liver will be verified by flow cytometry.

This reconstitution will provide the final confirmation for the involvement of type I and/or type II NKT cells in suppressing the F. tularensis-induced cytokine storm and death. Unfortunately, CD1d^{-/-} mice lacking both NKT cell subsets cannot be used as recipients due to the lack of the CD1d presenting molecule. The reconstitution of Jα18^{-/-} mice with type I iNKT cells will restore their susceptibility to F. tularensis to wild type levels. On the other hand, reconstitution of Jα18^{-/-} mice with type II NKT cells will augment the numbers of type II NKT cells still present in Jα18^{-/-} mice. Changes in the survival and cytokine storm may be observed due to increased numbers of type II NKT cells. Transfer of NKT cells into CD1d^{-/-} mice will serve as negative controls as they are non-functional in the absence of the presenting CD1d, allowing for cross activation within the transferred population. FIG. 5 suggests that type I iNKT cells are at least partly involved in protection from F. tularensis LVS challenge.

Development of an in vitro macrophage/NKT cell co-culture model of inflammation. In order to address the mechanism(s) by which NKT cells control the cytokine storm, an in vitro co-culture system has been adapted for this purpose.^{16,17} Immortalized wild type bone marrow-derived macrophages (BEI Resources) were infected with F. tularensis LVS for two hours followed by extensive washing to remove extracellular bacteria. Extracellular bacteria were not considered since it is known that F. tularensis must actively infect the cytoplasm of macrophages to elicit the inflammatory response. Indeed, heat-killed bacteria or bacteria deficient in the virulence master transcription factor MglA, which allows F. tularensis to escape the phagosome, fail to elicit inflammation. In response to in vitro infection, macrophages produced IL-1β and IL-6 (measured by ELISA) to initiate the inflammatory response. Optimization of this assay showed that prolonging infection time did not dramatically enhance IL-6 or IL-1μ production. Total NKT cells purified from wildtype splenocytes using Miltenyi microbeads were co-cultured with the infected macrophages at optimized effector:target ratios. In the presence of purified NKT cells, there was a reduction though not ablation of IL-6 and IL-1β (FIG. 7) thus recapitulating the reduction of inflammatory cytokines observed in vivo (see FIG. 4).

NKT cells control the cytokine storm by: (1) direct killing of F. tularensis or F. tularensis-infected cells preventing initiation of the inflammatory response, or (2) direct cytokine-mediated suppression of the inflammatory milieu.

2. Determine whether cytotoxic effector mechanisms limit the induction of the cytokine storm. The natural killing functions of NKT cells allow them to directly kill bacteria (e.g., granulysin) or host cells infected with bacteria (e.g., perforin). These actions limit the spread of the bacterium and could result in lower levels of inflammation. In addition, removal of host macrophages infected with bacteria results in fewer cells producing the inflammatory cytokines to initiate the response. Hence, it was considered whether NKT cells are suppressing the cytokine storm by reducing the amount of bacteria or bacteria-infected host cells. Though both subsets of NKT cells can express cytotoxic effector mechanisms, type I iNKT cells have predominately been considered to be cytotoxic in response to infection. Therefore, it was asked whether one, or both, NKT cell subsets could limit intracellular bacterial growth or kill F. tularensis-infected macrophages so as to prevent the induction of the cytokine storm.

2a. Do NKT cells directly kill intracellular F. tularensis preventing induction of inflammation? The level of bacterial burdens in the liver and spleen target organs of wildtype and CD1d^{-/-} mice infected with F. tularensis were compared. If NKT cells are directly killing bacteria, one expects to see a reduced bacterial burden in mice lacking NKT cells. Contrarily, no statistical difference was observed in the bacterial burdens of either organ between wildtype and NKT cell-deficient mice (FIG. 8). However, the growth rates of the bacteria in vivo could potentially vary between the two mouse strains. Therefore, wildtype and NKT cell-deficient mice will be inoculated with F. tularensis LVS and 5 animals sacrificed daily. The organ burdens of spleen, liver and lung will be used to plot an in vivo growth curve. This will reveal any differences in the growth of F. tularensis in the presence and absence of NKT cells.

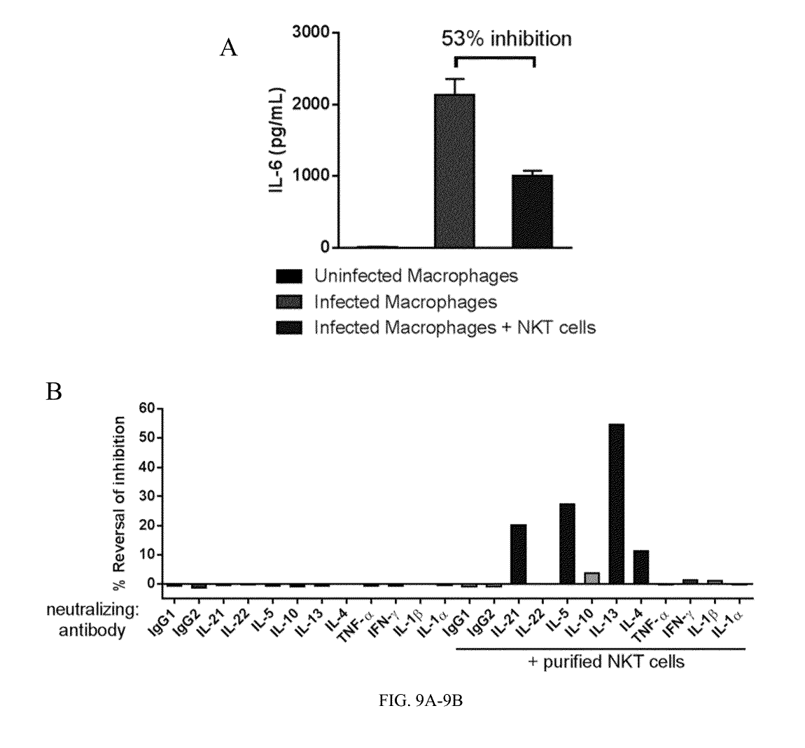

However, using whole animals only allows for examination of entire organ burdens. In order to investigate whether NKT cells could directly kill intracellular F. tularensis, co-cultured purified NKT cells with F. tularensis-infected macrophages were assessed in vitro. After 48 hours, the macrophages were gently lysed with 0.2% Saponin releasing the intracellular bacteria. Modified Mueller-Hinton broth containing 1 mCi/mL H³-uridine was added to the bacteria and the cultures were allowed to incorporate the radioactive uridine overnight. Bacteria were transferred to glass-fiber filters and the levels of radiation measured by scintillation counting. Uridine incorporation is proportional to the cfu count obtained for the same cultures (FIG. 9).^{16,66,67} Uninfected macrophages were used to control for incomplete lysis and subsequent incorporation of radioactive uridine. Though not significant (p=0.1), a trend was observed for the inhibition of intracellular F. tularensis growth in the presence of purified NKT cells (FIG. 9). This is suggestive that NKT cells might inhibit intracellular bacteria; thereby limiting initiation of inflammation and preventing the cytokine storm.

2b. Do NKT cells kill F. tularensis-infected macrophages? In the assay described above, killing only of the intracellular bacteria and killing of the macrophage harboring the bacterium cannot be distinguished. Therefore, co-culture assay described above was modified such that the macrophages were fluorescently labeled and plated in wells with a coverslip bottom. Following addition of purified NKT cells, the cells were incubated for 48 hours before addition of Propidium Iodide (PI) to label dead and dying cells. Using the BD Pathway Imager in the CSI Core Facility, images were obtained to detect incorporation of PI specifically into labeled macrophages undergoing apoptosis and not unlabeled NKT cells (FIG. 10). The percentage of macrophages undergoing apoptosis was compared between F. tularensis-infected macrophages cultured alone or in the presence of purified NKT cells. Though bacterial infection did induce some degree of apoptosis in macrophages, co-culture with NKT cells did not augment apoptosis (FIG. 11, p=0.9). This suggests that NKT cells do not suppress the cytokine storm by killing F. tularensis-infected macrophages thereby preventing them from initiating inflammation.

2c. Do cytotoxic mechanisms of NKT cell-mediated bacterial control suppress the cytokine storm. In order to determine the mechanism by which NKT cells cause inhibition of intracellular bacteria, neutralizing antibodies will be added to the in vitro co-culture.¹⁷ Of particular interest is granzyme A, granzyme A produced by γ9δ2 T cells is able to induce intracellular inhibition of mycobacteria.¹⁷ In addition, cytokines that result in the classical activation (e.g., IFN-γ, TNF-α) or alternative activation (e.g., IL-4 and IL-13) of macrophages will be initially targeted by neutralizing antibodies. Should NKT cell subsets be shown to induce apoptosis in F. tularensis-infected macrophages, potential targets would include: perforin, granzyme B, Fas/FasL, CD40/CD40L and TNF-α.

Using this in vitro data to focus on the most likely effector mechanism(s), the relevance of these mechanisms of NKT cell suppression in vivo will be confirmed. NKT cells will be purified from donors genetically deficient in the selected effector function and adoptively transferred into 10-15 Jα18^{-/-} recipients. This will result in deficiency of the effector mechanism specifically in the NKT cells of the reconstituted mice. F. tularensis LVS challenge of these animals should result in poor control of inflammation despite the physical presence of NKT cells compared with mice reconstituted with wild type NKT cells.

Based upon the data, it appears that NKT cells do not mediate the protection through their natural killing function (FIG. 11) but instead inhibits intracellular bacterial growth (FIG. 9) without killing macrophages.

3. Determine whether NKT cells suppress progression of the cytokine storm. While type I NKT cells are generally considered to be cytotoxic and produce pro-inflammatory cytokines, type II NKT cells are thought to have a regulatory role under conditions of inflammation and are antagonistic to pro-inflammatory iNKT cells.⁶⁻¹¹ However, emerging data suggest that, under certain conditions, type I iNKT cells can also suppress inflammation.¹¹⁻¹⁴ As shown in FIG. 4, mice deficient in NKT cells have higher serum levels of pro-inflammatory cytokines suggesting that NKT cells may directly suppress inflammation. Moreover, purified NKT cells co-cultured with F. tularensis-infected macrophages reduced IL-6 production in vitro (FIG. 7). Therefore, it is important to consider whether one or both NKT cell subsets can directly regulate the development of the cytokine storm.

3a. Antibody neutralization of potential effector functions in vitro. To test whether NKT cells directly regulate the cytokine storm, neutralizing antibodies directed against effector molecules expressed by NKT cells were added with to in vitro co-culture assays. Purified NKT cells were able to inhibit the production of IL-6 to 53% of control level (FIG. 12A). Addition of antibodies alone or isotype control antibodies had no effect on IL-6 production. However, in the presence of purified NKT cells, antibodies directed against IL-13, IL-5, IL-21 and IL-4 reversed the inhibition of IL-6 (i.e., there was more IL-6 in cultures with these antibodies) supplied by NKT cells by 11-55% (FIG. 12B). This effect now has been observed twice for IL-13, IL-5 and IL-21. In addition, it is important to determine whether these effector molecules are being produced by type I or type II NKT cells. Therefore, antibody neutralization will focus on effector molecules produced by independently purified type I and type II NKT cells. In addition, control experiments to demonstrate equal infection of macrophages and neutralization by the antibody will be conducted.

3b. Determine the contact requirement for NKT cell-mediated suppression of inflammation in vitro. It will be determined whether NKT cells are directly associated or activated by F. tularensis-infected macrophages. Macrophages will be pre-loaded with IL-6 SmartFlare RNA Detection probe (EMD Millipore) prior to infection with fluorescently labeled F. tularensis. Following co-culture with purified and fluorescently labeled NKT cells, the association between macrophages making IL-6 (SmartFlare⁺) and NKT cells will be determined temporally using the BD Pathway Confocal Imager. This will allow us to determine whether infected macrophages are producing IL-6 and whether NKT cells are directly interacting with these IL-6 producing macrophages.

It can then be determine whether the effector molecules act only locally on nearby cells or whether they can be secreted by the NKT cell and have activity more globally. This will be accomplished in vitro by transfer of culture supernatants from wells containing NKT cells to fresh wells containing only F. tularensis-infected macrophages. In recipient wells, only soluble effector molecules from the original co-cultures will be present. Suppression of inflammation after supernatant transfer would suggest a contact-independent suppressive mechanism and an endocrine modality. As controls, culture supernatant from wells lacking NKT cells, wells containing NKT cells alone, and culture supernatant from which the effector molecule has been absorbed using antibody will be transferred in parallel.

To complement the supernatant transfer experiments and in recognition that the effector molecule may be too dilute in culture supernatant, recombinant protein will be added to wells containing only F. tularensis-infected macrophages to mimic its secretion by NKT cells. Of necessity, excess recombinant protein will need to be added to obtain a similar local concentration as that secreted by NKT cells.

3c. Validation of potential mechanism(s) NKT cell use to suppress progression of the cytokine storm. If transfer of NKT cell-free supernatants or addition of neutralizing antibody demonstrates an endocrine mode of action for the responsible effector molecule, its systemic importance will be confirmed by injection of neutralizing antibody. Groups of 10 to 15 Jα18^{-/-} mice will be injected with neutralizing antibody one day prior to F. tularensis LVS challenge and again two and five days after inoculation. The survival and F. tularensis-induced cytokine storm in treated mice will be compared with that of isotype control injected animals.

To confirm the role of the identified NKT cell effector molecule(s), NKT cells will be purified from donors genetically deficient in the indicated effector function and adoptively transferred into 10 to 15 Jα18^{-/-} recipients. This will result in deficiency of the effector mechanism specifically in the NKT cells of the reconstituted mice. F. tularensis LVS challenge of these animals should result in poor control of inflammation despite the physical presence of NKT cells compared with mice reconstituted with wild type NKT cells.

The in vitro co-culture assay has been optimized such that NKT cell-mediated suppression of IL-6 and the reversal of that suppression can be observed.

Statistical analysis: Group sizes of 15 mice for survival analysis are sufficient to result in a statistical p-value of 0.05 with a power of 0.8 based upon the survival rates of B6 and CD1d^{-/-} mice by Mantel-Cox test. Subsequently, the Mantel-Cox test will be used to analyze the survival of all experimental groups. Mann-Whitney non-parametric tests will be utilized for comparisons of inflammation or the cytokine storm between groups of animals. For repeated sampling, the Wilcoxon matched pairs tests will be utilized. Two-way ANOVA with Tukey post-tests will be utilized for comparison amongst multiple groups or time points. In all cases a p-value<0.05 will be considered statistically significant.

Current data demonstrates that NKT cells are activated by the presence of F. tularensis, yet little is known regarding the role of NKT cells in controlling F. tularensis-induced inflammation. The data suggests that at least type I iNKT cells protect against development of the cytokine storm and subsequent death.

### References

1. Mares, C A, Ojeda, S S, Morris, E G, Li, Q & Teale, J M. Initial Delay in the Immune Response to Francisella tularensis Is Followed by Hypercytokinemia Characteristic of Severe Sepsis and Correlating with Upregulation and Release of Damage-Associated Molecular Patterns. Infection and Immunity 76, 3001-3010 (2008).

2. Sharma, J, Mares, C A, Li, Q, Morris, E G & Teale, J M. Features of sepsis caused by pulmonary infection with Francisella tularensis Type A strain. Microb Pathog 51, 39-47 (2011).

3. Rick, L C & Wu, T H. Animal models of Francisella tularensis infection. Ann NY Acad Sci 1105, 238-265 (2007).

4. Penn, R L. in Principles and Practice of Infectious Diseases (eds G. L. Mandell, J. E. Bennett, & R. Dolin) 2674-2685 (Elsevier: Churchill Livingstone, 2000).

5. D'elia, R V, Harrison, K, Oyston, P C, Lukaszewski, R A & Clark, G C. Targeting the “cytokine storm” for therapeutic benefit. Clinical and vaccine immunology : CVI 20, 319-327 (2013).

6. Ambrosino, E, Berzofsky, J A & Terabe, M. Regulation of tumor immunity: the role of NKT cells. Expert opinion on biological therapy 8, 725-734 (2008).

7. Ambrosino, E et al. Cross-regulation between type I and type II NKT cells in regulating tumor immunity: a new immunoregulatory axis. J Immunol 179, 5126-5136 (2007).

8. Berzofsky, J A & Terabe, M. A novel immunoregulatory axis of NKT cell subsets regulating tumor immunity. Cancer immunology, immunotherapy : CII 57, 1679-1683 (2008).

9. Berzofsky, J A & Terabe, M. NKT cells in tumor immunity: opposing subsets define a new immunoregulatory axis. J Immunol 180, 3627-3635 (2008).

10. Terabe, M et al. A nonclassical non-Valpha24Jalpha18 CD1d-restricted (type II) NKT cell is sufficient for down-regulation of tumor immunosurveillance. J Exp Med 202, 1627-1633 (2005).

11. Godfrey, D I & Kronenberg, M. Going both ways: immune regulation via CD1d-dependent NKT cells. J Clin Invest 114, 1379-1388 (2004).

12. Pilones, K A, Aryankalayil, J & Demaria, S. Invariant NKT cells as novel targets for immunotherapy in solid tumors. Clinical & developmental immunology 2012, 720803 (2012).

13. Godfrey, D I, Stankovic, S & Baxter, A G. Raising the NKT cell family. Nat Immunol 11, 197-206 (2010).

14. Watarai, H et al. Development and function of invariant natural killer T cells producing T(h)2- and T(h)17-cytokines. PLoS biology 10, e1001255 (2012).

15. De Pascalis, R, Taylor, B C & Elkins, K L. Diverse myeloid and lymphoid cell subpopulations produce gamma interferon during early innate immune responses to Francisella tularensis live vaccine strain. Infect Immun 76, 4311-4321 (2008).

16. Spencer, C T, Abate, G, Blazevic, A & Hoft, D F. Only a subset of phosphoantigen-responsive gamma9delta2 T cells mediate protective tuberculosis immunity. J Immunol 181, 4471-4484 (2008).

17. Spencer, C T et al. Granzyme A produced by gamma(9)delta(2) T cells induces human macrophages to inhibit growth of an intracellular pathogen. PLoS Pathog 9, e1003119 (2013).

18. Paget, C et al. Potential role of invariant NKT cells in the control of pulmonary inflammation and CD8+ T cell response during acute influenza A virus H3N2 pneumonia. J Immunol 186, 5590-5602 (2011).

19. De Santo, C et al. Invariant NKT cells reduce the immunosuppressive activity of influenza A virus-induced myeloid-derived suppressor cells in mice and humans. J Clin Invest 118, 4036-4048 (2008).

20. Tisoncik, J R et al. Into the eye of the cytokine storm. Microbiology and molecular biology reviews : MMBR 76, 16-32 (2012).

21. Stacey, A R et al. Induction of a Striking Systemic Cytokine Cascade prior to Peak Viremia in Acute Human Immunodeficiency Virus Type 1 Infection, in Contrast to More Modest and Delayed Responses in Acute Hepatitis B and C Virus Infections. Journal of virology 83, 3719-3733 (2009).

22. Sjostedt, A. Tularemia: history, epidemiology, pathogen physiology, and clinical manifestations. Ann NY Acad Sci 1105, 1-29 (2007).

23. Tarnvik, A, Eriksson, M, Sandstrom, G & Sjostedt, A. Francisella tularensis—a model for studies of the immune response to intracellular bacteria in man. Immunology 76, 349-354 (1992).

24. Ancuta, P, Pedron, T, Girard, R, Sandstrom, G & Chaby, R. Inability of the Francisella tularensis lipopolysaccharide to mimic or to antagonize the induction of cell activation by endotoxins. Infect Immun 64, 2041-2046 (1996).

25. Dreisbach, V C, Cowley, S & Elkins, K L. Purified lipopolysaccharide from Francisella tularensis live vaccine strain (LVS) induces protective immunity against LVS infection that requires B cells and gamma interferon. Infect Immun 68, 1988-1996 (2000).

26. Sandstrom, G, Sjostedt, A, Johansson, T, Kuoppa, K & Williams, J C. Immunogenicity and toxicity of lipopolysaccharide from Francisella tularensis LVS. FEMS Microbiol Immunol 5, 201-210 (1992).

27. Henry, T & Monack, D M. Activation of the inflammasome upon Francisella tularensis infection: interplay of innate immune pathways and virulence factors. Cell Microbiol 9, 2543-2551 (2007).

28. Mariathasan, S, Weiss, D S, Dixit, V M & Monack, D M. Innate immunity against Francisella tularensis is dependent on the ASC/caspase-1 axis. J Exp Med 202, 1043-1049 (2005).

29. Weiss, D S, Henry, T & Monack, D M. Francisella tularensis: activation of the inflammasome. Ann NY Acad Sci 1105, 219-237 (2007).

30. Mccaffrey, R L & Allen, L A. Francisella tularensis LVS evades killing by human neutrophils via inhibition of the respiratory burst and phagosome escape. J Leukoc Biol 80, 1224-1230 (2006).

31. Elkins, K L, Cooper, A, Colombini, S M, Cowley, S C & Kieffer, T L. In vivo clearance of an intracellular bacterium, Francisella tularensis LVS, is dependent on the p40 subunit of interleukin-12 (IL-12) but not on IL-12 p70. Infect Immun 70, 1936-1948 (2002).

32. Elkins, K L, Leiby, D A, Winegar, R K, Nacy, C A & Fortier, A H. Rapid generation of specific protective immunity to Francisella tularensis. Infect Immun 60, 4571-4577 (1992).

33. Chang, Y-J et al. Influenza infection in suckling mice expands an NKT cell subset that protects against airway hyperreactivity. The Journal of Clinical Investigation 121, 57-69 (2011).

34. Venken, K et al. Bacterial CD1d-Restricted Glycolipids Induce IL-10 Production by Human Regulatory T Cells upon Cross-Talk with Invariant NKT Cells. J Immunol (2013).

35. Crane, D D, Griffin, A J, Wehrly, T D & Bosio, C M. B1a Cells Enhance Susceptibility to Infection with Virulent Francisella tularensis via Modulation of NK/NKT Cell Responses. J Immunol 190, 2756-2766 (2013).

36. Schmitt, D M et al. Role of NK cells in host defense against pulmonary type A Francisella tularensis infection. Microbes and Infection.

37. Bendelac, A, Savage, P B & Teyton, L. The biology of NKT cells. Annu Rev Immunol 25, 297-336 (2007).

38. Florence, W C, Bhat, R K & Joyce, S. CD1d-restricted glycolipid antigens: presentation principles, recognition logic and functional consequences. Expert Rev Mol Med 10, e20 (2008).

39. Muindi, K et al. Activation state and intracellular trafficking contribute to the repertoire of endogenous glycosphingolipids presented by CD1d. Proc Natl Acad Sci USA 107, 3052-3057 (2010).

40. Banal, D C & Brenner, M B. CD1 antigen presentation: how it works. Nat Rev Immunol 7, 929-941 (2007).

41. Brigl, M & Brenner, M B. CD1: antigen presentation and T cell function. Annu Rev Immunol 22, 817-890 (2004).

42. Van Kaer, L. alpha-Galactosylceramide therapy for autoimmune diseases: prospects and obstacles. Nat Rev Immunol 5, 31-42 (2005).

43. Zhou, D et al. Lysosomal glycosphingolipid recognition by NKT cells. Science 306, 1786-1789 (2004).

44. Kinjo, Y et al. Recognition of bacterial glycosphingolipids by natural killer T cells. Nature 434, 520-525 (2005).

45. Mattner, J et al. Exogenous and endogenous glycolipid antigens activate NKT cells during microbial infections. Nature 434, 525-529 (2005).

46. Sriram, V, Du, W, Gervay-Hague, J & Brutkiewicz, R R. Cell wall glycosphingolipids of Sphingomonas paucimobilis are CD1d-specific ligands for NKT cells. Eur J lmmunol 35, 1692-1701 (2005).

47. Kinjo, Y et al. Natural killer T cells recognize diacylglycerol antigens from pathogenic bacteria. Nat Immunol 7, 978-986 (2006).

48. Tupin, E et al. NKT cells prevent chronic joint inflammation after infection with Borrelia burgdorferi. Proc Natl Acad Sci USA 105, 19863-19868 (2008).

49. Kobayashi, E, Motoki, K, Uchida, T, Fukushima, H & Koezuka, Y. KRN7000, a novel immunomodulator, and its antitumor activities. Oncol Res 7, 529-534 (1995).

50. Cardell, S et al. CD1-restricted CD4+ T cells in major histocompatibility complex class II-deficient mice. J Exp Med 182, 993-1004 (1995).

51. Godfrey, D I, Macdonald, H R, Kronenberg, M, Smyth, M J & Van Kaer, L. NKT cells: what's in a name? Nat Rev Immunol 4, 231-237 (2004).

52. Jahng, A et al. Prevention of autoimmunity by targeting a distinct, noninvariant CD1d-reactive T cell population reactive to sulfatide. J Exp Med 199, 947-957 (2004).

53. Chang, D H et al. Inflammation-associated lysophospholipids as ligands for CD1d-restricted T cells in human cancer. Blood 112, 1308-1316 (2008).

54. Godfrey, D I et al. Antigen recognition by CD1d-restricted NKT T cell receptors. Seminars in immunology 22, 61-67 (2010).

55. Miyamoto, K, Miyake, S & Yamamura, T. A synthetic glycolipid prevents autoimmune encephalomyelitis by inducing TH2 bias of natural killer T cells. Nature 413, 531-534 (2001).

56. Yu, KOA et al. Modulation of CD1d-restricted NKT cell responses by using N-acyl variants of a-galactosylceramides. Proceedings of the National Academy of Sciences of the United States of America 102, 3383-3388 (2005).

57. Goff, RD et al. Effects of Lipid Chain Lengths in a-Galactosylceramides on Cytokine Release by Natural Killer T Cells. Journal of the American Chemical Society 126, 13602-13603 (2004).

58. Ragin, M J, Sahu, N & August, A. Differential regulation of cytokine production by CD1d-restricted NKT cells in response to superantigen staphylococcal enterotoxin B exposure. Infect Immun 74, 282-288 (2006).

59. Kato, S, Terabe, M & Berzofsky, J. Liver sulfatide-reactive type II NKT cells recognize endogenous phospholipids. (INC9P.445). The Journal of Immunology 192, 188.184 (2014).

60. Gordy, L E et al. IL-15 regulates homeostasis and terminal maturation of NKT cells. J Immunol 187, 6335-6345 (2011).

61. Coquet, J M et al. Diverse cytokine production by NKT cell subsets and identification of an IL-17-producing CD4-NK1.1-NKT cell population. Proc Natl Acad Sci USA 105, 11287-11292 (2008).

62. Terashima, A et al. A novel subset of mouse NKT cells bearing the IL-17 receptor B responds to IL-25 and contributes to airway hyperreactivity. J Exp Med 205, 2727-2733 (2008).

63. Hess, P R et al. Selective deletion of antigen-specific CD8+ T cells by MHC class I tetramers coupled to the type I ribosome-inactivating protein saporin. Blood 109, 3300-3307 (2007).

64. Vincent, B G et al. Toxin-Coupled MHC Class I Tetramers Can Specifically Ablate Autoreactive CD8+ T Cells and Delay Diabetes in Nonobese Diabetic Mice. The Journal of Immunology 184, 4196-4204 (2010).

65. Spangrude, G J. in Current Protocols in Immunology (John Wiley & Sons, Inc., 2001).

66. Worku, S & Hoft, D F. Differential effects of control and antigen-specific T cells on intracellular mycobacterial growth. Infect Immun 71, 1763-1773 (2003).

67. Worku, S & Hoft, D F. In vitro measurement of protective mycobacterial immunity: antigen-specific expansion of T cells capable of inhibiting intracellular growth of bacille Calmette-Guerin. Clinical infectious diseases : an official publication of the Infectious Diseases Society of America 30 Suppl 3, S257-261 (2000).

1. A method for modulating a cytokine response comprising administering conditioned NKT cells to a subject.

2. A method to treat Francisella tularensis infection comprising administering an activator of NKT cells or a condition NKT cell to a subject infected by Francisella tularensis.

3. A method to treat cytokine storm from infectious diseases comprising administering a combination of recombinant IL-4, IL-5, IL-10, IL-13, or IL-21 or any combination therein.
